(19) United States
(12) Patent Application Publication
(10) Pub. No.: US 2008/0317255 A1
(43) Pub. Date: Dec. 25, 2008

(54) Audio Transducer Component

(75) Inventors: John Cozens, Nokia (FI); Nicholas Zacharov, Tampere (FI)

Correspondence Address: SQUIRE, SANDERS & DEMPSEY L.L.P., 8000 TOWERS CRESCENT DRIVE, 14TH FLOOR, VIENNA, VA, 22182-6212, US

(73) Assignee: NOKIA CORPORATION, Espoo (FI)

(21) Appl. No.: 11/885,146

(22) PCT Filed: Feb. 25, 2005
(86) PCT No.: PCT/FI05/00125

§ 371 Date: Jun. 26, 2008

(51) Int. Cl. H04R 9/06; A61F 11/06

(52) U.S. Cl. 381/71.6; 381/182

(57) ABSTRACT

An audio transducer component comprises at least two independent voice coils, each voice coil associated with a diaphragm and an acoustic cavity. The audio transducer component further comprises magnetic means for driving the at least two independent voice coils with independent signals.

### FIELD OF THE INVENTION

The invention relates to audio transducer components. The present invention also relates to electronic devices, such as speakers, earpieces, communication devices, and so on, comprising such audio transducer components.

### BACKGROUND OF THE INVENTION

An electronic device may be provided with speaker means. Such electronic devices may comprise mobile communication devices or terminals, such as user equipment (UE), a mobile station (MS), a cellular phone, a personal digital assistant (PDA) and so on, or other electronic devices, such as a laptop computer, a sound recorder or a sound player.

A communication device may be used for tasks such as for making and receiving phone calls, for receiving and sending data from and to the network and for experiencing multimedia content or otherwise using multimedia services. In addition to speaker means, a communication device may be provided with an antenna, display, camera means, and so on. The operation of a communication device may be controlled by means of an appropriate user interface such as control buttons, voice commands and so on. Furthermore, a communication device is provided with a processor entity and a memory means.

In current and future electronic devices, in particular mobile terminals, more and more audio transducers may need to be placed within a device, for example to include and improve hands-free and earpiece audio. Traditionally, as components are required, they are added separately. For example, multi-channel or stereo hands-free product feature may require two large hands-free speaker components to be available in a device. This may be difficult to achieve in a small volume product. Another example may comprise three dimensional (3D) or stereo dipole or virtual sound reproduction for a product to be employed for effective creation of spatial sound for games, user interface (UI) sound or multi-channel (e.g. 5.1 channel) sound reproduction in a compact mobile device. Furthermore, hands-free and earpiece audio reproduction may require a hands-free speaker and an earpiece receiver component.

U.S. Pat. No. 5,548,657 in the name of Fincham relates to a compound loudspeaker drive unit, which is a dual cone speaker component.

U.S. Pat. No. 6,622,817 in the name of Bachmann et al. relates to panel loudspeakers working according to the bending wave principle, wherein two acoustic panels are arranged at a distance in relation to each other by providing elements connecting both acoustic panels and by arranging drivers on the sides of the acoustic panels facing each other.

Patent Application US2003/0048920 A1 in the name of Van Halteren et al. relates to an electro-acoustic transducer with two diaphragms and two coils, which have a common magnetic circuit having a first and a second gap wherein a magnetic field is established. In Van Halteren, the double diaphragm transducer can be operated in two modes, the two diaphragm-coil systems being electrically coupled in phase or out of phase. The transducer of Van Halteren can be operated by deflecting the first and second diaphragms in the same direction or in opposite directions upon providing, simultaneously, the same electrical signal to the first and second coils.

There is a need for improved solutions for including speaker components in electronic devices, in particular in portable or mobile electronic devices. It might be desired to combine separate transducer components into one package, which preferably should occupy less space or volume than said separate components originally.

### SUMMARY OF THE INVENTION

In accordance with an aspect of the invention, there is provided an audio transducer component comprising at least two independent voice coils, each voice coil associated with a diaphragm and an acoustic cavity; and magnetic means for driving the at least two independent voice coils with independent signals.

In an embodiment, the magnetic means may comprise a single magnet.

In an embodiment, the magnetic means may comprise at least two magnets. In an embodiment, a first magnet may serve concurrently two independent voice coils and a second magnet may serve at least one further independent voice coil.

In an embodiment, the independent signals may comprise a signal of at least one of sub-speech frequencies, speech frequencies, narrow band speech, wideband speech and full audible frequency range. In an embodiment, the independent signals may comprise at least one of a stereo signal, a three-dimensional sound signal, a monophonic signal and an active noise control signal.

In an embodiment, said acoustic cavities associated with each of the at least two independent voice coils may be independent from each other.

In an embodiment, the at least two voice coils, together with the associated diaphragms and acoustic cavities, may be placed back to back.

In an embodiment, the magnetic means may comprise one of an annular, disk and block shaped magnet. In an embodiment, the magnetic means may comprise a linearly polarised magnet. In an embodiment, the magnetic means may comprise a radially polarised magnet.

In an embodiment, the audio transducer component may further comprise at least one of an independent port for each acoustic cavity for air venting to free air and an independent connecting structure for each acoustic cavity. In an embodiment, the at least one independent port may comprise at least one of a side vent, a front vent and a back vent.

In an embodiment, the at least two voice coils may be acoustically isolated from each other.

In an embodiment, one of the at least two voice coils and the associated diaphragm and acoustic cavity may be configured to act as a microphone and another of the at least two voice coils and the associated diaphragm and acoustic cavity are configured to act as a speaker. In an embodiment, the speaker may be configured to reproduce an earpiece signal and an active noise controlled signal for noise cancellation. In an embodiment, the audio transducer component may further comprise a further independent voice coil associated with a diaphragm and an acoustic cavity, wherein the further voice coil and the associated diaphragm and acoustic cavity may be acting as an active noise controlled speaker for noise cancellation.

In an embodiment, at least two of the at least two voice coils and the associated diaphragm and acoustic cavity may each be acting as a speaker.

In accordance with a further aspect of the invention, there is provided an electronic device comprising an audio transducer component.

The electronic device may comprise one of an earpiece, a hands-free device, a user equipment, a mobile station, a cellular phone, a game terminal, a remote control, a camcorder, a personal digital assistant, a laptop computer, a sound recorder and a sound player.

Various embodiments and variations of the invention shall become clear from the following detailed description and the attached claims.

### BRIEF DESCRIPTION OF THE DRAWINGS

The invention will now be described in further detail, by way of example only, with reference to the following examples and accompanying drawings, in which:

FIG. 1 shows an example of a communication device in which the embodiments of the invention may be implemented;

FIG. 2 shows a cross section view of a typical miniature electro-dynamic speaker component;

FIG. 3 shows a cross section view of an implementation comprising two speaker components;

FIG. 4 shows a cross section view of a further implementation comprising two speaker components;

FIG. 5 shows a cross section view of an embodiment of the invention;

FIG. 6 shows a cross section view of a further embodiment of the invention;

FIG. 7 shows a cross section view of a further embodiment of the invention;

FIG. 8 shows a cross section view of a further embodiment of the invention;

FIG. 9 shows a cross section view of a further embodiment of the invention;

FIG. 10 shows a cross section view of a further embodiment of the invention; and

FIG. 11 shows a perspective view of a component according to the embodiment described in schematic form in FIG. 5.

### DETAILED DESCRIPTION OF PREFERRED EMBODIMENTS

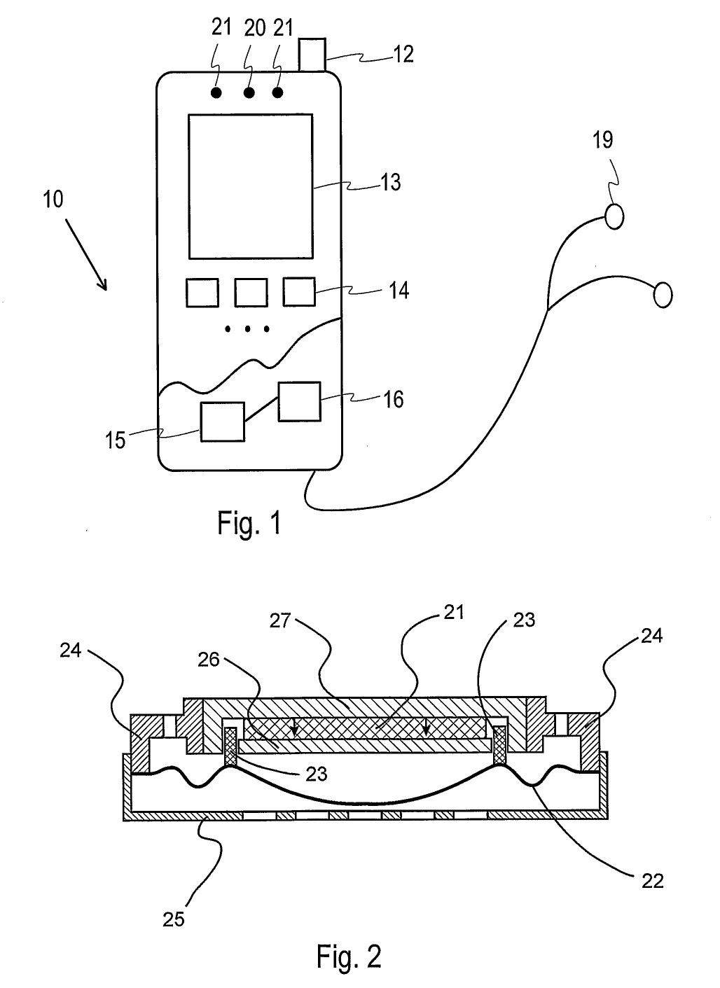

FIG. 1 shows an example of a communication device 10 comprising an antenna 12 for radio reception and transmission. The communication device 10 is provided with a display 13 and control buttons 14. Furthermore, a processor entity 15, electrical memory means 16, first speaker means 19 and second speaker means 20 are provided.

FIG. 1 shows only one exemplifying communication device in which the embodiments of the invention may be implemented. The communication device of FIG. 1 has a form of a mobile station. It shall be appreciated that the type of the communication device may differ substantially from what is shown in FIG. 1. The radio reception and transmission means may as well be built in the casing of the communication device or arranged in another appropriate manner. The control buttons of any appropriate form may be positioned in an appropriate manner depending on the communication device type, size and use, for example.

Electronic devices where embodiments of the invention may be implemented may comprise mobile communication devices or terminals or other electronic devices. Examples may comprise, but are not limited to, user equipment (UE), a mobile station (MS), a cellular phone, a game terminal (portable or domestic), a remote control, a camcorder, a personal digital assistant (PDA), a laptop computer, a sound recorder or a sound player, and any other electronic devices comprising speaker means or other audio transducer components. The speaker means may be a speaker or loudspeaker, for example an speaker referred to as 20 together with more than one port or vent, referred to as 21 in FIG. 1, radiating sound for e.g. stereo sound reproduction applications, or a headphone, for example as the speaker means referred to as 19 in FIG. 1, or the like.

FIG. 2 shows a typical miniature electro dynamic speaker component, which may be used for both hands-free and earpiece audio. The speaker component typically comprises a linearly polarised magnet 21, a diaphragm 22, a voice-coil 23, a frame 24, a cover 25 and iron parts 26, 27.

It has now been found that functionality of two or more audio transducer components might be combined into a single component package using an integrated technical solution. With designs illustrated in embodiments of the invention, two or more hands-free speakers or other audio transducer components can be combined together into one package that may be smaller than such two or more hands-free speakers separately. Said one package comprises at least two independent acoustic generators, such as a voice coil with diaphragm, and a single magnet serving at least two acoustic generators. Each voice coil of the acoustic generators is driven with an independent signal.

Embodiments of the invention may reduce the amount of area required by the speaker. Embodiments of the invention may allow improved pickup or reproduction of stereo signals also from compact devices. Packages according to embodiments of the invention may occupy the same or less volume, less mass and compromise of fewer components for assembly. Vital for 3D sound reproduction, the component solutions could be created so that two speaker channels are acoustically identical which a highly valued characteristics for ensuring successful and plausible 3D sound algorithm implementations.

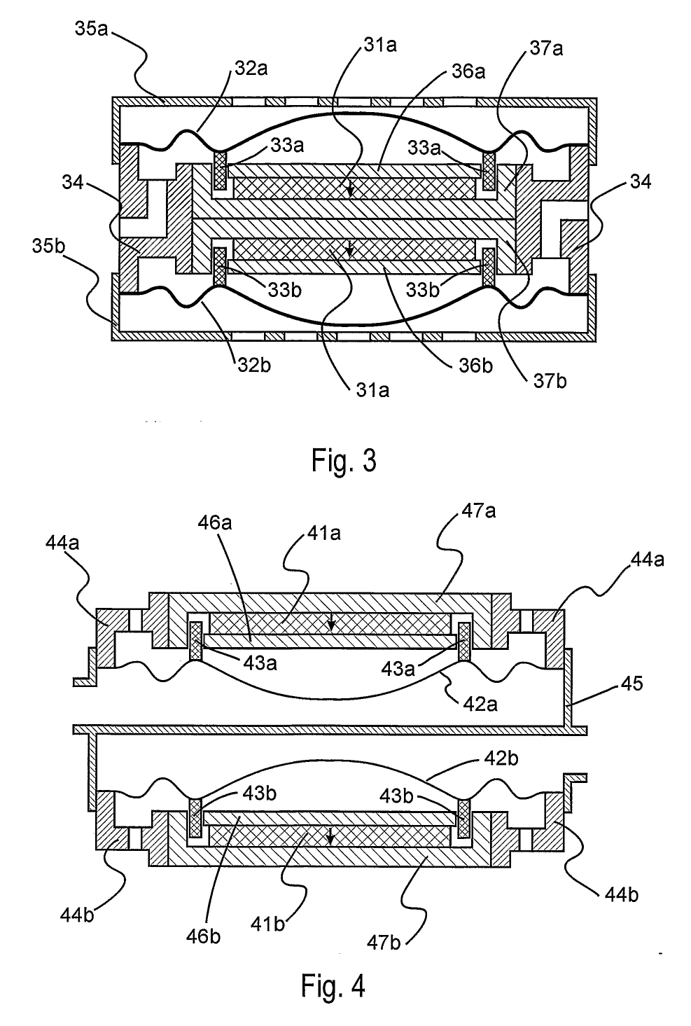

FIG. 3 shows a possible implementation of a stereo hands-free speaker component, where two individual speaker components similar to the speaker components of FIG. 2 are placed back to back. The speaker component of FIG. 3 comprises two linearly polarised magnets 31a, 31b, two diaphragms 32a, 32b, two voice-coils 33a, 33b, covers 35a, 35b and iron parts 36a, 36b, 37a, 37b. The frame part 34 is modified to enable a join to be made and designed such that air movement from behind each vibrating diaphragm can vent into free air, but remain isolated within the speaker component.

FIG. 4 shows another possible implementation of a stereo hands-free speaker component, where the two individual speaker components are placed front to front. The speaker component of FIG. 4 comprises two linearly polarised magnets 41a, 41b, two diaphragms 42a, 42b, two voice-coils 43a, 43b, two frames 44a, 44b and iron parts 46a, 46b, 47a, 47b. The cover 45 is modified to enable a join to be made and designed such that air movement from front of each vibrating diaphragm can vent into free air, but remain isolated within the component.

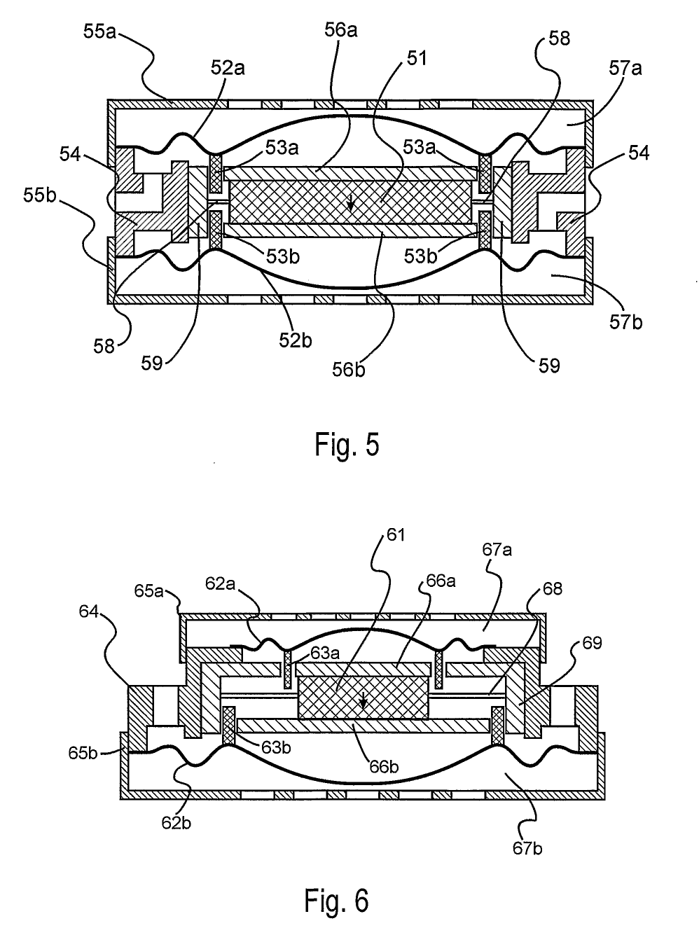

FIG. 5 shows an embodiment of the invention for a stereo speaker component. The speaker component of FIG. 5 comprises two diaphragms 52a, 52b, two voice-coils 53a, 53b, a frame 54, covers 55a, 55b and iron parts 56a, 56b, 59. Here the number of magnets has been reduced to one linearly polarised magnet 51 serving both voice coils 53a, 53b and diaphragms 52a, 52b. This may save on cost and weight compared to separate magnets for each acoustic generator. The thickness of the component may also be reduced. The voice coils 53a, 53b and diaphragms 52a, 52b, as well as the air chambers or acoustic cavities 57a, 57b are independent of each other. An acoustic isolation 58 between the two voice coils may or may not be used.

FIG. 6 shows a further embodiment of a simple combination of an earpiece and hands-free speaker. The speaker of FIG. 6 is a modification to the embodiment of FIG. 5, by shrinkage in the voice coil 63a and diaphragm 62a sizes. As an example, such shrinkage in a linear dimension could be, for example, 25 to 50% in embodiments of the invention. The earpiece-speaker component of FIG. 6 comprises one linearly polarised magnet 61 two diaphragms 62a, 62b, two voice-coils 63a, 63b, a frame 64, covers 65a, 65b, iron parts 66a, 66b, 69 and separated air chambers or acoustic cavities 67a, 67b. As in the implementation of FIG. 5, an acoustic isolation 68 between the two systems may or may not be used. In addition, this design could be used as an enhanced hands-free speaker, with low and high frequency production units.

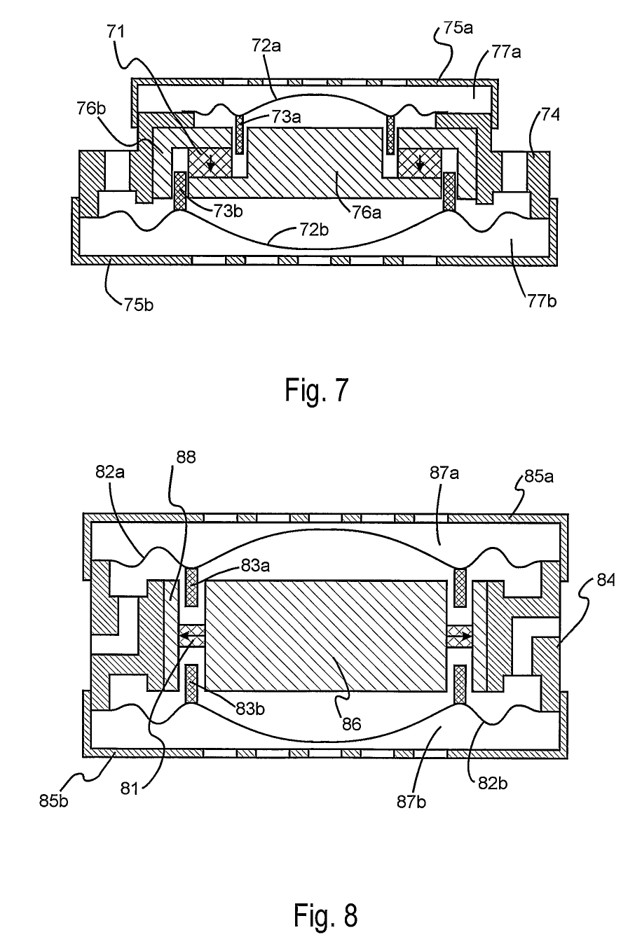

FIG. 7 shows an alternative design for the same functionality than the implementation of FIG. 6. In FIG. 7, an annular shaped magnet 71 is used and linearly polarised. The earpiece-speaker component of FIG. 7 comprises the magnet 71, two diaphragms 72a, 72b, two voice-coils 73a, 73b, a frame 74, covers 75a, 75b, iron parts 76a, 76b and separated air chambers or acoustic cavities 77a, 77b. The speaker means 19 of FIG. 1 could be an example of a use for the embodiment of FIG. 7. The upper, shorter diaphragm 72a may provide a speaker for earpiece and the lower, longer diaphragm 72b may provide a microphone for sound pickup, or visa versa. Two such earphones could be used for obtaining stereo or binaural sound recording and reproduction.

FIG. 8 shows a further embodiment of a single magnet system allowing hands-free combination designs of a stereo loudspeaker component. The embodiment of FIG. 8 employs a radially polarised single magnet 81. The speaker component of FIG. 8 comprises the magnet 81, two diaphragms 82a, 82b, two voice-coils 83a, 83b, a frame 84, covers 85a, 85b, iron parts 86, 88 and separated air chambers or acoustic cavities 87a, 87b.

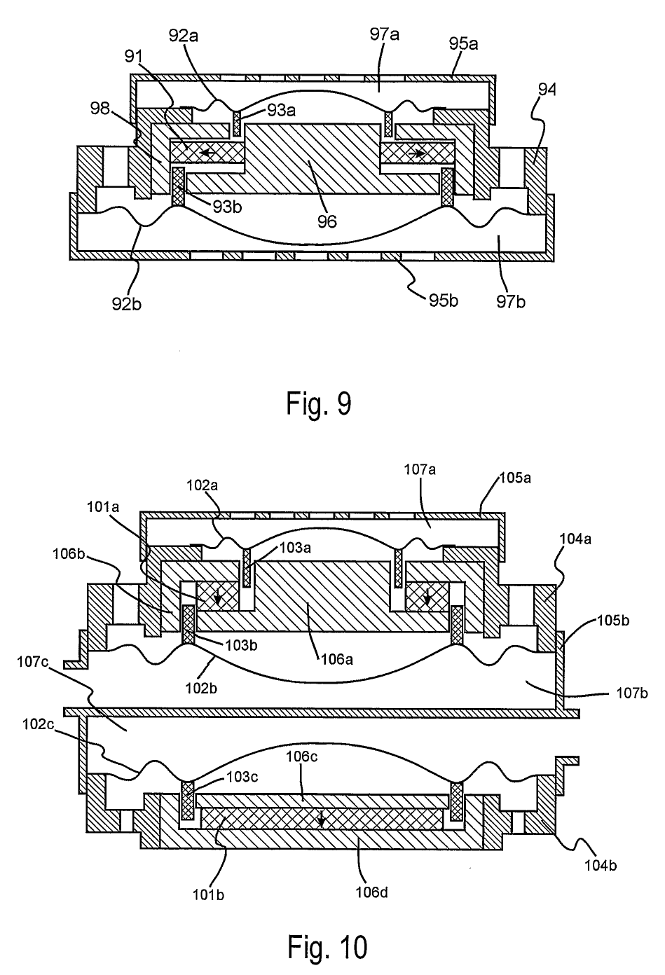

FIG. 9 shows a further embodiment of a radially polarised magnet 91 for a hands-free and/or earpiece component. The component of FIG. 9 comprises the magnet 91, two diaphragms 92a, 92b, two voice-coils 93a, 93b, a frame 94, covers 95a, 95b, iron parts 96, 98 and separated air chambers or acoustic cavities 97a, 97b.

Embodiments shown in FIGS. 7-9 may be preferred implementations. FIGS. 8 and 9 illustrate a highly efficient magnetic design of a single component transducer employing a single, radially polarised magnet 81, 91. This design may provide high flux density across the coils 83, 93, as the magnetic flux across the gap is provided from the whole of the magnet 81, 91. This may be advantageous compared to the embodiment of FIG. 5, where the flux is half the magnets 51 flux density for each diaphragm 52a, 52b. Whilst the design FIGS. 8 and 9 may be relatively expensive due to the radially polarised magnet 81, 91, it may provide a truly symmetrical design. This may be important for 3D or virtual sound applications. Furthermore, the component of embodiments shown in FIGS. 8 and 9 can be made very compact due to the motor structure. This design can be tailored for either a hands-free/earpiece application or a multi-channel/stereo sound reproduction application.

FIGS. 3-9 show component combinations for just two components. However, more than two components could be combined in accordance with embodiments of the invention. In addition, electro-dynamic speaker designs have been shown, but it is appreciated that the invention of combining more than one diaphragm in a single component is not restricted to only electro-dynamic designs.

FIG. 10 shows an embodiment of a three-channel hands-free and/or earpiece component. The component of FIG. 10 comprises two linearly polarised magnets 101a, 101b, three diaphragms 102a, 102b, 102c, three voice-coils 103a, 103b, 103c, two frames 104a, 104b, covers 105a, 105b, iron parts 106a, 106b, 106c, 106d, and separated air chambers or acoustic cavities 107a, 107b, 107c. The component may comprise at least one of an independent port for each acoustic cavity for air venting to free air, as shown by references 107b and 107c in FIG. 10, and an independent connecting structure, illustrated by references 105a and 105b, for each acoustic cavity.

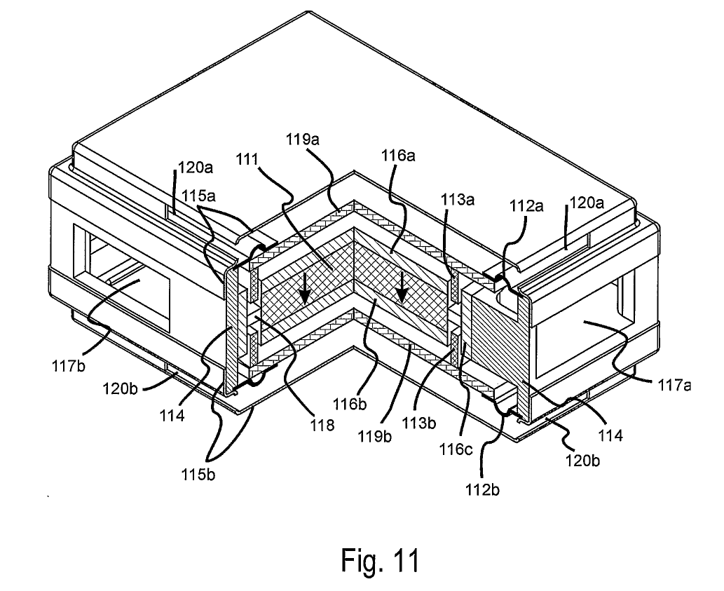

FIG. 11 shows a practical embodiment of a component similar to the described in schematic form in FIG. 5. The component of FIG. 11 comprises a circular disk linearly polarised magnet 111, diaphragm parts 112a, 112b, 119a, 119b, circular voice coils 113a, 113b, iron parts 116a, 116b, 116c, a frame 114, and covers 115a, 115b. Independent air cavities 117a, 117b vent from one side of each diaphragm to the outside through the sides of the component. Air from the other side of each diaphragm vents through slots 120a, 120b cut into the cover parts 115a,115b.

Furthermore, the embodiments shown in FIGS. 3-10 comprise air venting of a speaker component to the front and back. A speaker component may comprise side vents for air venting, instead or in addition to the front and back vents by means of the design of the cover parts of the component.

In embodiments of the invention, one of the at least two voice coils and the associated diaphragm and acoustic cavity may act as a microphone for sound pickup and another of the at least two voice coils and the associated diaphragm and acoustic cavity may act as a speaker.

In embodiments, the independent signals driving the at least two independent voice coils may comprise a signal of different frequencies. Appropriate frequencies may comprise sub-speech frequencies (about 20 to 500 Hz), speech frequencies (about 500 to 3000 Hz) and full audible frequency range (about 20 to 20 000 Hz). Defining in an alternative way, appropriate frequencies may comprise narrow band speech about from 300 to 3400 Hz, wideband speech about from 100/150 to 7000 Hz and audio bandwidth from 20 to 20 000 Hz. The audio bandwidth may be even wider for super resolution audio and may then be defined as an audible bandwidth. In an embodiment, the independent signals may comprise at least one of a stereo signal, a three-dimensional sound signal, a monophonic signal and an active noise control signal.

In the following, some non-limiting examples are given to illustrate different usages of designs of the invention. Reference is made to FIGS. 3-11.

In a first embodiment, a two-way internal hands-free (IHF) speaker may be implemented using the component shown in one of FIGS. 6, 7 and 9. An IHF speaker may allow a group of people in the vicinity of the device to hear and/or to take part in a phone conversation. In this embodiment, shorter of the diaphragms, shown as an upper diaphragm 62a, 72a, 92a in FIGS. 6, 7 and 9, respectively, may be used for a low frequency (LF) mono signal. Longer of the diaphragms, shown as a lower diaphragm 62b, 72b, 92b in FIGS. 6, 7 and 9, respectively, may be used for a high frequency (HF) mono signal.

In a second embodiment, a stereo IHF speaker may be implemented using the component shown in one of FIGS. 3, 4, 5, 8 and 11. In this embodiment, one of the diaphragms, for example the upper diaphragm 32a, 42a, 52a, 82a, 112a, may be used for a left channel signal. Another of the diaphragms, for example the lower diaphragm 32b, 42b, 52b, 82b, 112b, may be used for a right channel signal.

In a third embodiment, a combined IHF speaker and earpiece may be implemented using the component shown in one of FIGS. 6,7 and 9. In this embodiment, the shorter diaphragm 62a, 72a, 92a may act as a mono IHF speaker, or visa versa. The longer diaphragm 62b, 72b, 92b may act as a mono earpiece speaker.

In a fourth embodiment, a headphone may be implemented using the component shown in one of FIGS. 6,7 and 9. In this embodiment, the shorter diaphragm 62a, 72a, 92a may act as an earpiece mono speaker of the headphone. The longer diaphragm 62b, 72b, 92b may act as a microphone. The diaphragms may be interchanged. In a further embodiment, two of said components may be used in headphones, one for the left and one for the right ear, whereby a binaural recording and reproduction may be obtained.

In a fifth embodiment, an active noise controlled (ANC) earpiece may be implemented using the component shown in FIG. 7. In this embodiment, the shorter diaphragm 72a may act as an earpiece mono speaker. The longer diaphragm 72b may act as an ANC speaker for noise cancellation. The diaphragms may be interchanged.

In an alternative implementation of the fifth embodiment, an active noise controlled (ANC) earpiece may be implemented using the component shown in FIG. 10. In this embodiment, the shorter diaphragm 102a may act as an earpiece mono speaker. The longer diaphragm 102b served by the same magnet 106a than the diaphragm 102a may act as an ANC speaker for noise cancellation. Furthermore, the other longer diaphragm 102c, which may be served by the separate magnet 106c, may act as a microphone for control of ANC algorithm. The diaphragms may be interchanged.

In a sixth embodiment, an alternative ANC earpiece may be implemented using the component shown in one of FIGS. 6,7 and 9. In this embodiment, the shorter diaphragm 62a, 72a, 92a may act as an earpiece mono speaker for reproduction of both earpiece and ANC signal. The longer diaphragm 62b, 72b, 92b, may act as a microphone for control of ANC algorithm. The diaphragms may be interchanged.

In a seventh embodiment, a three-channel speaker may be implemented using the component shown in FIG. 10. In this embodiment, the shorter diaphragm 102a may act as a center speaker. The longer diaphragm 102b may act as a left speaker. Furthermore, the other longer diaphragm 102c may act as a right speaker. The diaphragms may be interchanged.

In an eighth embodiment, a combined earpiece and stereo IHF speaker may be implemented using the component shown in FIG. 10. In this embodiment, the shorter diaphragm 102a may act as an earpiece mono speaker. The longer diaphragm 102b may act as a left IHF speaker. Furthermore, the other longer diaphragm 102c may act as a right IHF speaker.

Compared to two or more individual components, embodiments of the invention may provide lower cost due to a single magnet. Furthermore, lower weight and smaller foot print surface area that is needed on the printed circuit board (PCB) receiving the component may be achieved. Compact and modular component concept may allow rapid design and implementation of multi-channel/stereo speaker into mobile terminals. Symmetrical acoustic designs, which may be obtained in some embodiments, may be highly suited to the requirements of 3D or virtual sound reproduction requirements.

Although the invention has been described in the context of particular embodiments, various modifications are possible without departing from the scope and spirit of the invention as defined by the appended claims. It should be appreciated that whilst embodiments of the present invention have mainly been described in relation to mobile communication devices, embodiments of the present invention may be applicable to other types of electronic devices comprising speaker means or other audio transducer components.

1-20. (canceled)

21. An audio transducer component comprising at least two independent voice coils, each voice coil associated with a diaphragm and an acoustic cavity; and magnetic means for driving the at least two independent voice coils with independent signals, wherein said acoustic cavities associated with each of the at least two independent voice coils are independent from each other.

22. The audio transducer component according to claim 21, wherein the magnetic means comprises a single magnet.

23. The audio transducer component according to claim 21, wherein the magnetic means comprises at least two magnets.

24. The audio transducer component according to claim 23, wherein a first magnet serves concurrently two independent voice coils and a second magnet serves at least one further independent voice coil.

25. The audio transducer component according to claim 21, wherein the independent signals comprise a signal of at least one of sub-speech frequencies, speech frequencies, narrow band speech, wideband speech and full audible frequency range.

26. The audio transducer component according to claim 25, wherein the independent signals comprise at least one of a stereo signal, a three-dimensional sound signal, a monophonic signal and an active noise control signal.

27. The audio transducer component according to claim 21, wherein the at least two voice coils, together with the associated diaphragms and acoustic cavities, are placed back to back.

28. The audio transducer component according to claim 21, wherein the magnetic means comprises one of an annular, disk and block shaped magnet.

29. The audio transducer component according to claim 21, wherein the magnetic means comprises a linearly polarised magnet.

30. The audio transducer component according to claim 21, wherein the magnetic means comprises a radially polarised magnet.

31. The audio transducer component according to claim 21, further comprising at least one of an independent port for each acoustic cavity for air venting to free air and an independent connecting structure for each acoustic cavity.

32. The audio transducer component according to claim 31, wherein the at least one independent port comprises at least one of a side vent, a front vent and a back vent.

33. The audio transducer component according to claim 21, wherein the at least two voice coils are acoustically isolated from each other.

34. The audio transducer component according to claim 21, wherein one of the at least two voice coils and the associated diaphragm and acoustic cavity are configured to act as a microphone and another of the at least two voice coils and the associated diaphragm and acoustic cavity are configured to act as a speaker.

35. The audio transducer component according to claim 34, wherein the speaker is configured to reproduce an earpiece signal and an active noise controlled signal for noise cancellation.

36. The audio transducer component according to claim 34, further comprising a further independent voice coil associated with a diaphragm and an acoustic cavity, wherein the further voice coil and the associated diaphragm and acoustic cavity are acting as an active noise controlled speaker for noise cancellation.

37. The audio transducer component according to claim 21, wherein at least two of the at least two voice coils and the associated diaphragm and acoustic cavity are each acting as a speaker.

38. An electronic device comprising an audio transducer component according to claim 21.

39. The electronic device according to claim 38, comprising one of an earpiece, a hands-free device, a user equipment, a mobile station, a cellular phone, a game terminal, a remote control, a camcorder, a personal digital assistant, a laptop computer, a sound recorder and a sound player.
